(19) United States
(12) Patent Application Publication
(10) Pub. No.: US 2011/0184250 A1
(43) Pub. Date: Jul. 28, 2011

(54) EARLY WARNING METHOD AND SYSTEM FOR CHRONIC DISEASE MANAGEMENT

(75) Inventors: Steven P. Schmidt, Wellesley, MA (US); Santosh Ananthraman, Allison Park, PA (US); Thomas H. Smith, Winchester, MA (US)

(73) Assignee: Asthma Signals, Inc., Woburn, MA (US)

(21) Appl. No.: 13/011,394

(22) Filed: Jan. 21, 2011

Related U.S. Application Data
(60) Provisional application No. 61/297,151, filed on Jan. 21, 2010.
(60) Provisional application No. 61/298,740, filed on Jan. 27, 2010.

(51) Int. Cl. A61B 5/00

(52) U.S. Cl. 600/300

(57) ABSTRACT

A computer-implemented method and system are provided for assisting a plurality of patients manage chronic health conditions. The method, for each patient, comprises: (a) receiving information from the patient or a member of a patient care network on an expected patient activity at a given future time period; (b) determining expected transient local ambient conditions in the patient's surroundings during the expected patient activity at the given future time period; (c) predicting health exacerbations for the patient using a stored computer model of the patient based on a desired patient control set-point range, the expected patient activity, and the expected transient local ambient conditions; and (d) proactively sending a message to the patient or a member of the patient care network before the given future time period, the message alerting the patient or a member of the patient care network of the predicted health exacerbations for the patient and identifying one or more corrective actions for the patient to avoid or mitigate the predicted health exacerbations.

### CROSS REFERENCE TO RELATED APPLICATIONS

The present application claims priority from U.S. Provisional Patent Application Ser. No. 61/297,151, filed on Jan. 21, 2010, entitled HEALTH MANAGEMENT SYSTEM AND METHOD and U.S. Provisional Patent Application Ser. No. 61/298,740, filed on Jan. 27, 2010, entitled HEALTH MANAGEMENT SYSTEM AND METHOD, both of which are hereby incorporated by reference.

### BACKGROUND

The present application relates to an early warning method and system for chronic disease management.

The Milken Institute Center for Health Economics study: “An Unhealthy America: The Economic Burden of Chronic Disease—Charting a New Course to Save Lives and Increase Productivity and Economic Growth” released in 2007, quantified chronic disease current and future treatment costs, as well as the economic losses for business, across all 50 states. Researchers tracked seven chronic diseases (such as asthma) and found the impact on the U.S. economy to be $1.3 trillion annually, including lost productivity totals of $1.1 trillion and $277 billion for treatment.

Asthma is a chronic lung disease characterized by inflammation, bronchoconstriction, and an increase in mucus production. It is a widespread public health problem that has increased in the past two decades in the United States. In 2007, an estimated 34 million (11.5%) in the U.S. population had lifetime asthma and 22.9 million (7.7%) had current asthma. In 2006, the asthma hospitalization rate for all ages was 14.9 per 10,000 U.S. residents, accounting for approximately 444,000 hospitalizations. There were 3,884 asthma-related deaths in the U.S. in 2005 with a mortality rate of 1.3 per 100,000 residents.

Asthma affects more children than any other chronic disease and is one of the most frequent reasons for hospital admissions among children. Anyone can get asthma, but children are especially vulnerable. Asthma is twice as common among children as adults. Asthma is one of the most common chronic childhood diseases. Over six million asthma sufferers in the Unites States are under age 18. Asthma is the third ranking cause of hospitalization for children and one of the leading causes of school absenteeism. A total of 12.8 million school days are missed each year because of asthma. According to Allergy and Asthma Foundation of America, the estimated annual cost of asthma is nearly $19.7 billion, including nearly $10 billion in direct health care costs (mostly for hospitalizations) and $8 billion for indirect costs such as lost earnings due to illness or death. Asthma is the fourth leading cause of work absenteeism and diminished work productivity for adults, resulting in nearly 12 million missed or less productive workdays each year.

While asthma cannot be cured, it generally can be controlled. However, self-management of asthma for pediatric patients is difficult without continuous vigilance from their care network. It has been widely discussed that better education, self-help tools and better support from the care network can especially help children better manage their asthma on a day to day basis, thereby reducing asthma exacerbations and the subsequent need to use emergency hospitalization resources.

Disclosed herein are methods and systems for near-real time monitoring of continuous life situation behaviors of a given patient with a chronic disease and the transient local ambient conditions in the patient's milieu to predict and provide early warning in the form of corrective actions to the patient. These actions will potentially help mitigate the occurrence of catastrophic situations that would necessitate the patient's seeking emergency medical care or alter normal life activities, degrading the quality of life for the patient and their extended real world care network.

### BRIEF SUMMARY OF THE DISCLOSURE

In accordance with one or more embodiments, a computer-implemented method is provided for assisting a plurality of patients manage chronic health conditions. The method, for each patient, comprises: (a) receiving information from the patient or a member of a patient care network on an expected patient activity at a given future time period; (b) determining expected transient local ambient conditions in the patient's surroundings during the expected patient activity at the given future time period; (c) predicting health exacerbations for the patient using a stored computer model of the patient based on a desired patient control set-point range, the expected patient activity, and the expected transient local ambient conditions; and (d) proactively sending a message to the patient or a member of the patient care network before the given future time period, the message alerting the patient or a member of the patient care network of the predicted health exacerbations for the patient and identifying one or more corrective actions for the patient to avoid or mitigate the predicted health exacerbations.

In accordance with one or more further embodiments, an early warning system is provided for assisting a plurality of patients manage chronic health conditions. The early system comprises a computer system communicating with client devices operated by the plurality of patients over a communications network. For each patient the computer system is configured to: (a) receive information from the patient or a member of a patient care network on an expected patient activity at a given future time period; (b) determine expected transient local ambient conditions in the patient's surroundings during the expected patient activity at the given future time period; (c) predict health exacerbations for the patient using a stored computer model of the patient based on a desired patient control set-point range, the expected patient activity, and the expected transient local ambient conditions; and (d) proactively transmit a message to the patient or a member of the patient care network before the given future time period, the message alerting the patient or a member of the patient care network of the predicted health exacerbations for the patient and identifying one or more corrective actions for the patient to avoid or mitigate the predicted health exacerbations.

### BRIEF DESCRIPTION OF THE DRAWINGS

FIG. 1 is a simplified block diagram illustrating operation of an early warning system for chronic disease management in accordance with one or more embodiments.

FIG. 2 is an exemplary asthma control assessment procedure screen displayed on a mobile device operated by a user in accordance with one or more embodiments.

FIG. 3 is a simplified flow chart illustrating the operation of patient-optimized detection, trending, and training firmware for cough detection.

FIGS. 4A and 4B are tables illustrating exemplary belief type survey questions and analysis in accordance with one or more embodiments.

FIG. 5 is a table illustrating exemplary execution probabilities for mitigation action for various belief types in accordance with one or more embodiments.

FIG. 6 is a simplified block diagram illustrating an early warning system for chronic disease management in accordance with one or more embodiments.

FIG. 7 is a table illustrating an example of message scoring in accordance with one or more embodiments.

FIG. 8 is a schematic diagram illustrating a profile updating scheme in accordance with one or more embodiments.

FIG. 9 shows screenshots on a user mobile device illustrating exemplary action messages sent to a patient.

FIG. 10 is a simplified block diagram illustrating a model predictive control methodology in accordance with one or more embodiments.

FIG. 11 is a graph illustrating an example of an asthma probability range and graph estimate of exacerbation in accordance with one or more embodiments.

FIG. 12 is a table illustrating an example of ozone scoring heuristics in accordance with one or more embodiments.

FIG. 13 is a graph of an exemplary functional look-up in accordance with one or more embodiments.

FIG. 14 shows screenshots on a user mobile device illustrating an example of an action feed to a patient in accordance with one or more embodiments.

FIG. 15 is a table illustrating example of an augmented message for a teen with atopic asthma in accordance with one or more embodiments.

FIG. 16 shows graphs illustrating examples of predicted plan adherence in accordance with one or more embodiments.

Like or identical reference numbers are used to identify common or similar elements.

### DETAILED DESCRIPTION

The present application relates to a health management system and method to help people with chronic diseases (e.g., asthma, COPD, cystic fibrosis, multiple sclerosis, and depression) or difficult chronic disease treatment plans (e.g., HCV retroviral drug treatment regimens) better manage their diseases/treatments and maintain healthy, ambulatory lifestyles. As will be discussed in further detail below, in accordance with one or more embodiments, a near real-time system and method are provided for monitoring transient local conditions (e.g., local air quality, allergen levels, temperature, prevailing atmospheric conditions, in home environment) and patient behaviors (e.g., physical activity, treatment adherence) in continuous life situations to predict and thereby provide early warning to the patients with the goal of helping prevent health exacerbations and symptom control breakouts.

Such health exacerbations and symptom control breakouts can be catastrophic for individuals living with chronic or long-term health problems such as asthma. In accordance with one or more embodiments, the system generates electronically delivered audio and/or visual alerts to proactively indicate a probable impending exacerbation or control breakout during planned daily life activities involving a transient local condition or daily life behavior activities. The alerts identify health burden variables and appropriate mitigation actions that are most likely related to the predicted exacerbation or breakout such that appropriate control actions can be taken by the patient or his or her care network (e.g., the patient's parent or care guardian) to avoid the occurrence of the breakout or to mitigate the severity of the breakout occurrence, and avoid exacerbation events and resulting outcomes such as emergency room visits or hospitalization.

Scoring of alert validity and behavior modification success, along with longitudinal data for an individual, can be used to further individualize and optimize the variable weighting for a patient's health-monitoring model using various learning and inference techniques. The system also provides the capability for gross visualization and trending at both the individual and the location-population (community) level. The individual longitudinal trends and reports provide patients and their care guardians with information to identify problematic situations. The location-population (community) level assessments provide early warning and report back transient changes for pre-emptive actions to be taken by concerned groups such as healthcare administrators, insurance companies, and governmental agencies.

Patients diagnosed with chronic illness often must incorporate doctor-recommended lifestyle changes and self-management regimens into their routine life situations. Patients who are able to determine when to apply these doctor-recommended lifestyle changes and successfully follow these recommended lifestyle changes rigorously and comply with their self-management regimen are likely to have better individual health outcomes. Better health outcomes for chronically ill patients result in better quality of life for patients and their loved ones and the more efficient use of health care resources spent on chronic disease.

Self-management regimens can be difficult to adopt and maintain. This is especially true for pediatric populations or in diseases where immediate feedback of behaviors to health state is not perceived by the patient. Failure to adhere to doctor-recommended behaviors frequently results in severe non-immediate penalties such as delayed compromised physiological function or acute exacerbations, e.g., asthma attacks in the case of asthma, resulting in urgent visits to hospital emergency rooms. This breakdown occurs since patients do not know when an intervention is likely to be needed and which intervention is most likely to positively affect a patient's health status in real world settings to regularly promote actions that keep their health in a safe “green zone” and not let it drift into a problematic “yellow zone” or in the worst case, precipitously drop into the hazardous “red zone.”

It would be beneficial to provide patients with a near-real time interactive tool to help monitor real world settings, the health effect of patients' planned activities, and self-monitor adherence to their health plan regimen by recommending contemporaneously relevant, timely corrective actions that would promote their health and safety in daily life situations, thereby improving their total quality of life.

As will be described in further detail below, a system for predicting and managing the health behavior and treatment plan actions for a patient includes a remote management system that communicates with devices operated by a plurality of users (patients, their respective care network, or by concerned groups) over a communications network.

In accordance with one or more embodiments, a remote assessment and management system accesses data from a data store, which stores assessment data elements indicative of patient planned activities, medical conditions, and condition-relevant exacerbation triggers associated with patients, who can be geographically dispersed. In one or more embodiments, the system includes a decision support system working in tandem with an event processing engine, which contemporaneously applies a predictive model to a first set of selected assessment data elements to produce current health assessment measures for a patient against the patient's personal best or literature predicted best measures. The decision support system utilizes historical, current, and predicted local trigger burden, personal performance range, their doctor-recommended treatment plan, and health measures to produce a customized alert action plan applicable for patient daily life scenarios. This alert action plan is regularly updated based on the current, aggregate, actual, and predicted burden measures. The system also includes a rule base and a profile base for establishing a patient-specific model for the patient consistent with the patient's doctor-recommended treatment action plan. The system also includes an event-processing engine, which generates the timely patient-specific alert actions based upon assessment of data feeds against the model.

In accordance with one or more embodiments, an individualized, patient specific predictive model is selected from a pool of trigger burden algorithms based upon an assessment of condition severity, condition symptom triggers, physician-supplied condition or treatment management plan, location specific conditions and putative behaviors, selected patient and family assessment tools, and a feedback history of model goodness of fit to actual health and symptoms.

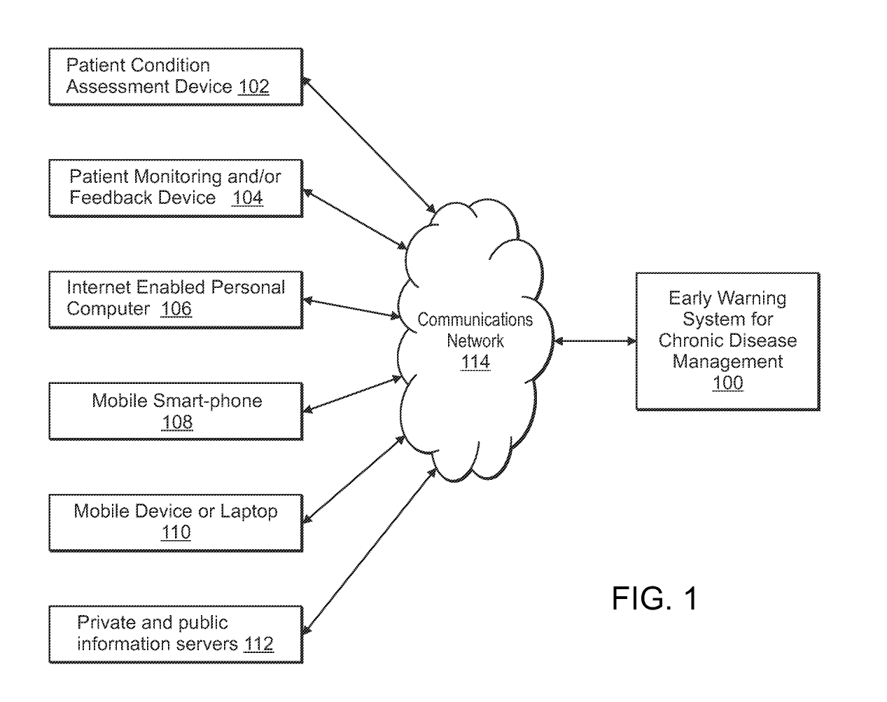

FIG. 1 is a simplified block diagram illustrating operation of a chronic disease management system 100 in accordance with one or more embodiments. The system 100 can be implemented in a computer server system and accessed by a variety of client devices 102, 104, 106, 108, 110 operated by users. The client devices can access the system 100 over a communications network 114. The network 114 may be any combination of networks, including without limitation the Internet, a local area network, a wide area network, a wireless network, and a cellular network. As discussed in further detail below, the client devices 102, 104, 106, 108, 110 comprise a variety of devices including personal computers and portable communications devices such as smart phones. The computer server system may comprise one or more physical machines, or virtual machines running on one or more physical machines. In addition, the computer server system may comprise a cluster of computers or numerous distributed computers that are connected by a network.

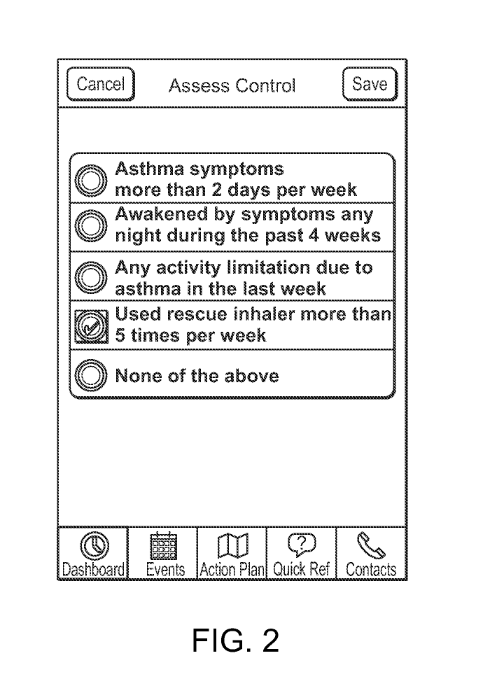

The user devices can include a patient condition assessment device 102, which can be a device that records the results of surveys (e.g., NAEPP Asthma control assessment survey, a portion of which is shown in the smartphone screen shot of FIG. 2), expert assessment (doctor diagnosis), or physiological function measurement devices (e.g., Spirometry FEV1: forced expiratory volume in one second). The device 102 can include a visual output display and/or or one-way or two-way electronic communication capabilities (either analog radio or digital).

The patient monitoring and/or feedback device 104 can be a medical monitoring device such as cough and wheeze detection devices, phones and other devices with microphones, portable cameras, pedometers and motion detectors, medication compliance monitors, game consoles, behavior monitoring applications (e.g., monitoring communication patterns), devices to record the results of a survey (e.g., PHQ-9 Questionnaire) and other devices configured to perform the monitoring functions. The device 104 has the capability for one-way or two-way communication (either analog radio or digital).

For example, in accordance with one or more embodiments, a recorded voice survey from a patient can be collected periodically, stored, analyzed, and interpreted for unhealthy sounds and changes from a healthy baseline recording for such things as wheeze, amplitude, pause between words, and comparison to personal best for a phrase. The wheeze, amplitude, pause, and other patterns are analyzed against known indicators for progression to an exacerbation event. The longitudinal analysis is performed to detect degradation of health based upon an individual's best recording, also analyzed for tell tales such as wheeze, pause, pattern, duration of sound, etc. Techniques such as the variants of the Hidden-Markov-Model algorithm can be used for sound detection, and multi-variable, nonlinear pattern recognition methods such as neural networks can be used to detect pattern variations.

In accordance with one or more embodiments, one or more device inputs are used to develop an individual profile for normal and abnormal activity. The delta between normal and pathology-induced detected changes is established using a personal best normal longitudinal baseline for the individual using both literature lookup of normal device reports and feedback from the person (e.g., I am feeling good) to establish the normal baseline. Literature and feedback (e.g., I had symptoms and/or a disease episode) is used to identify behavior, activity, voice, cough frequency, sleep pattern, etc., to establish pathology induced variation from normal. The baseline and disease characterized deviations from baseline are used to establish a personal probabilistic model and their conditional dependencies to determine the likelihood that a measured delta is indicative of a future disease exacerbation event.

Devices inputs can be comprised of any device that delivers information on patient elective behavior (telephone activity, gaming activity, exercise, etc.), physiological measurements (Spirometry, vital signs, sound, sleep, cough, wheeze, etc.), care community measures (frequency of contact, duration of contacts, network effectiveness of interaction, etc.), and measures of disease management practices (drug adherence, doctor's visits, etc.). Device measurements are generally collected in a real world setting. In most cases, clinical institution measurements are scored separately since they differ in quality and in many cases, are supervised by a care professional. As such, non-real world and real world measurements are catalogued as separate pools of data for the development of and analysis by the personal models.

The patient monitoring and/or feedback device can comprise a cough monitoring device. This ambulatory monitoring device includes a microphone, analytic firmware to detect, analyze, and interpret cough frequency similar to the methods used to develop the Leicester Cough Monitor. The device communicates pertinent analytic results comprising cough frequency via the network using radio frequency and/or Bluetooth transmission, and raw data download via Bluetooth, wireless, or USB connection to a computer. The device may be recharged, e.g., via USB connection or induction plate. The analytic firmware can be updated to use the appropriate algorithms for an individual demographic based, e.g., on age, disease with abnormal cough frequency components (such as asthma, bronchitis, rhinitis, Sarcoidosis, COPD, Cystic Fibrosis, and others), abnormal cough frequency profile and duration, and alert thresholds.

The cough detection algorithm, which can be a Leicester Cough Algorithm based on Hidden Markov Models to characterize the spectral properties of a time varying pattern. The selective detection of cough, which comprises a sound profile spotting approach similar to that used in speech recognition in which the objective is to detect the occurrence of a particular sound pattern in a sequence of continuous sound. Using a very weak microphone pendent resting on the chest cavity, rather than the sensitive microphone in the Leicester Cough Monitor, produced excellent cough feature detection relative to non-cough signal (e.g., car door slamming), allowing for automated scoring of cough and non-cough sounds.

The device may use a default set of detection algorithms based upon literature cough frequency per disease per human demographic profile (age, size, sex, etc.). Cough comprises individual explosive sounds collected with a relative amplitude and frequency for each person over time. This data can be used to train the statistical detection model of the characteristics of cough sounds and audio background sounds. Additionally, the firmware may be updated with further refined detection, thresholds, and analysis routines from the computer system.

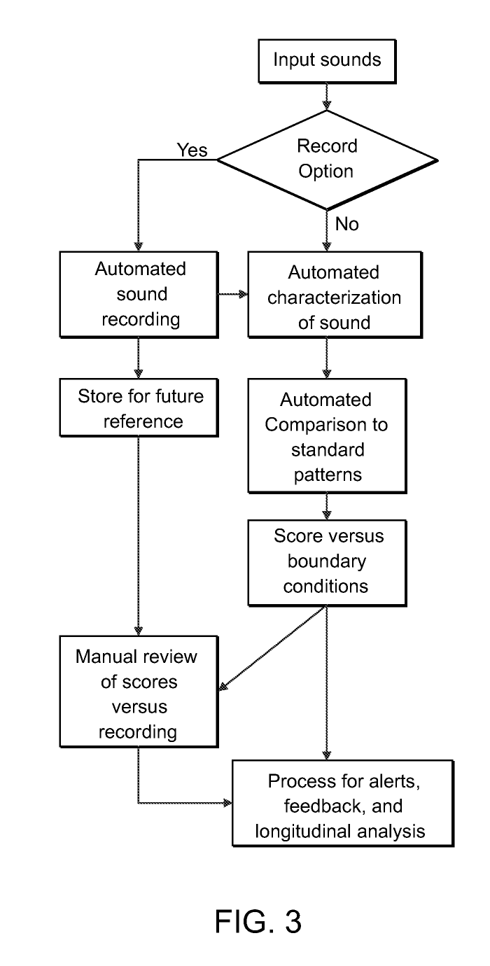

The coughs per person per time unit are measured and compared to control and chronic cough patients for healthy cough range and an alert range indicative of a loss of healthy range cough frequency. FIG. 3 is a simplified flow chart illustrating the operation of patient-optimized detection, trending, and training firmware for cough detection.

In accordance with one or more embodiments, a telephone can also be used as part of a monitoring and assessment system. A landline or cell phone can be used as an input device for detecting symptoms, surrogate markers, or other biometric measurement (symptoms, surrogate markers, or other biometric measurement herein called biometrics) for the purpose of detecting leading indicators for exacerbation events. Inputs measurement analytics comprise ah hoc detection of biometrics and for longitudinally analyzing changes from individual or peer population norms.

For example, a 20 second audio capture of breathing and talking a standard assessment sentence can be analyzed, e.g., for amplitude, pitch, shortness of breath, cadence of speech, and compared with population and individual benchmarks as a leading indicator of worsening symptoms for that individual. This type of detection may be especially useful where the patient is a child and the primary care guardian cannot physically observe or listen to the child for worsening symptoms. If the child patient, e.g., went to a sleepover, using this remote audio or visual assessment is simpler than trying to train the sleepover parent all the observation skills needed by someone watching for worsening symptoms.

The phone input comprises segments of audio, video, motion, or activity, and this information is sent to the remote system for analysis. A voice analysis using pitch and amplitude perturbation features, and a set of measures of the harmonic-to-noise ratio are extracted from the transmitted speech files. Features are extracted and classified using known methods including those developed by http://www.voxpilot.com. These feature sets are used to test and train automatic classifiers, employing the method of Hidden Markov modeling and Linear Discriminant Analysis. Amplitude perturbation features proved most robust in channel transmission.

An example of how assessment and monitoring survey is used to establish execution probabilities, communication types, and message/support priorities by care network roles is illustrated in connection with the belief survey described below.

The six archetypes beliefs surveyed for (and their distribution in the US population) are:

Dependable (18%):

Core Belief: The doctor knows best and I will do the right thing for my health

Need the doctor to tell me what to do

Default activity: Positive and proactive toward treatment plan

Expert—convince me first (20%):

Core Belief: No one is doing anything to help me

Need to be convinced treatment is effective before adopting it

Default activity: Research alternatives and share information

Superstitious—mind over matter (15%):

Core Belief: I am positive and living healthy, I am OK

Need to realize that just living better is not enough

Default activity: Focus on maintaining positive life changes (and ignore the hard facts)

Denier—entrenched doubter (21%)

Core Belief: There is really nothing I can do, so I'll ignore it

Need to realize the consequences of putting off managing the disease

Default activity: Avoid facing the disease

Rebel—authority adverse, live for today (15%):

Core Belief: Authority figures are using me and I won't live long anyway

Need someone they trust to set them straight and give them hope for the future

Default activity: Just struggling to get through today

Overwhelmed—(11%)

Core Belief: I can't handle the sustained process alone

Need to know there is support to help me sustain the treatment plan

Default activity: struggle to incorporate additional behaviors into daily life

The beliefs of patients and parents of patients greatly determine treatment plan adoption and adherence. For example, in asthma only about 40% of the overall population fill prescriptions given them at the end of an emergency room (ER) visit. However, 90% of “Dependable” belief types fill prescriptions given to them in the ER.

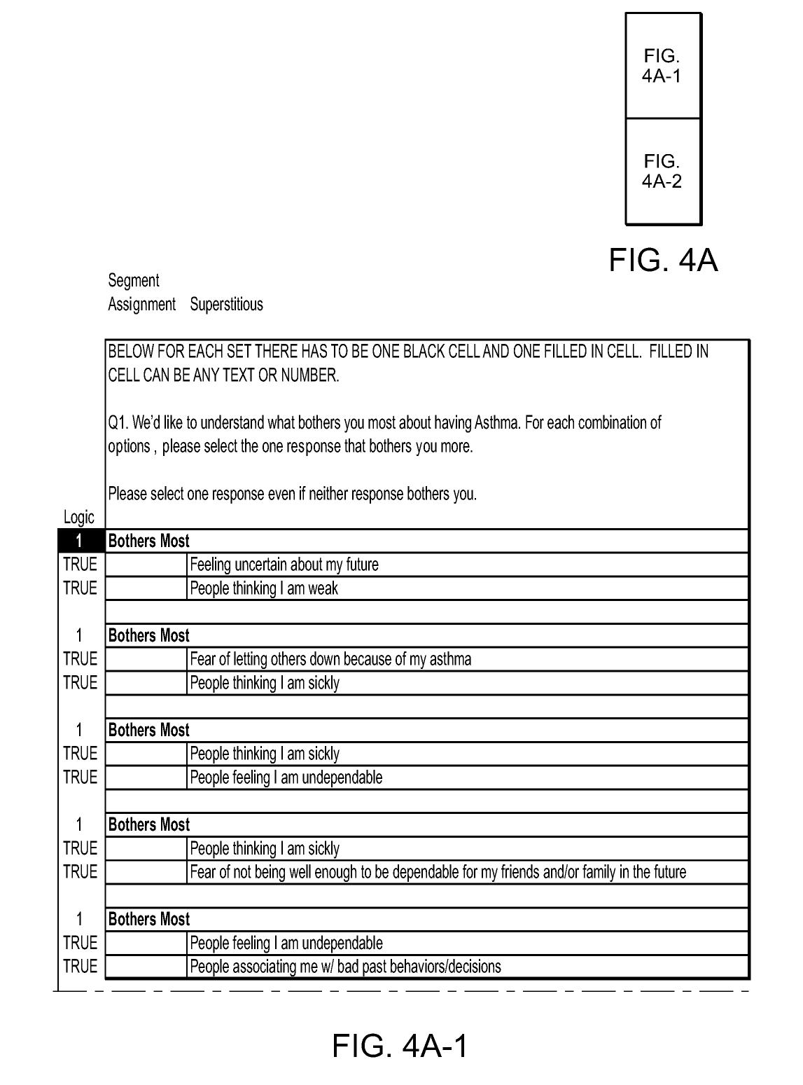

Knowing the belief type of an individual allows us to set the probability of people acting upon a directive message and subsequently scoring the patient's probable health state due to said execution of a directive. Additionally, knowing the belief type helps select the best type of communication message and support needed for patients and families of patients to be educated about and sustain adoption of treatment plans. The tables in FIGS. 4A and 4B illustrate a belief type segmentation survey and analysis for people with asthma using Wards Linear Discriminant Function in suboptimal situations.

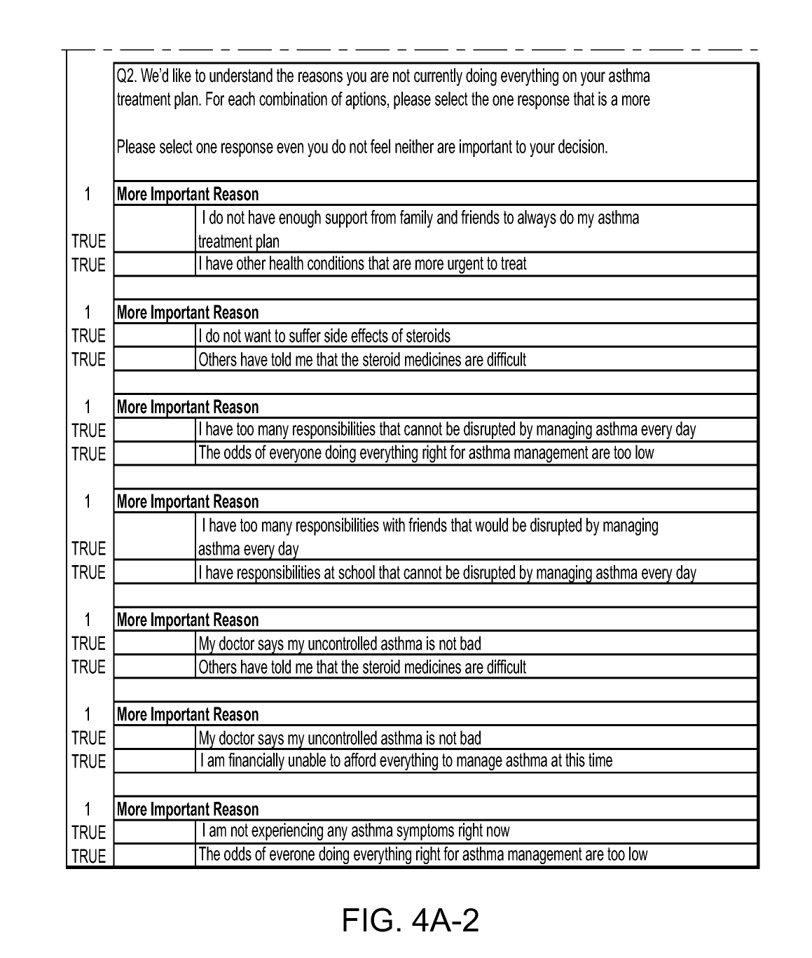

Using belief typing can motivate people to adopt and to sustain adherence to a treatment plan. This information can be used to score the probability that an action recommendation is executed in the absence of direct feedback from the patient or the appropriate person in the patient's care network. FIG. 5 is a table illustrating belief type application to execution probabilities for heuristic scoring of likely compliance to recommended mitigation actions.

A simple example of tailored content comprises an action message that includes a reference along with the action directive for the “Expert” type person. This ability to easily become knowledgeable about the details behind a directive increases the likelihood of executing the directive by 35%. In the case of the “Overwhelmed” type person, (e.g., single mom for a child with asthma), adding an advocate or helper into his or her care network to help with execution, increases the likelihood of execution by 40%.

Action upon probability of mitigation execution can also comprise a differential number, frequency and breadth of action messages to a patient and/or the number of people in a patient's care network who receive the action message. For example, a low probability of execution by a teenager with asthma causes the system to send the directive to the parent as well as the patient.

The user device 106 can comprise a variety of computer devices including Internet enabled devices such as personal computers, game consoles, smartphones, personal digital assistants, etc. that can be used to access patient data such as personal schedules and calendars and personal diaries, health histories and logs from phones, home, school and other environments frequented by the patient. These devices have the capability for one-way or two-way communication to either access event data feeds from the patient or to report back alert action feeds to the patient.

The mobile smart-phone device 108 can comprise an audio and/or visual asynchronous or synchronous communication device such as a phone with voice, text, and/or smart-phone capabilities. It can also be a wireless computer tablet or a wireless gaming console. These devices have the capability for one-way or two-way communication to either access event data feeds from the patient or to report back alert action feeds to the patient.

The mobile device or laptop 110 comprises an audio and/or visual asynchronous or synchronous communication device such as a wireless laptop, a computer tablet, or a wireless gaming console. These devices have the capability for one-way or two-way communication to either access event data feeds from the patient or to report back alert action feeds to the patient.

The private or public information servers 112 can comprise a variety of sources of private and public raw data, mined information, visualizations, and trend charts. The servers can be used to access transient ambient data obtained by monitoring local conditions comprising local air quality, allergen levels, temperature, prevailing atmospheric conditions or geographic information system. Transient ambient data can also include macro level trends such as exacerbation spikes and disease outbreaks and other related catastrophes.

The private or public information server devices can include local or remotely stored applications for accessing patient community information such as social care networks, calendars, and perform reporting and communications functions.

Each of the user devices 102, 104, 106, 108, 110 includes a network interface comprising an asynchronous or synchronous connection to the communications network 114 through one-way or two-way alpha-numeric paging services, voice services, Voice over Internet Protocol (VoIP), dialup and Broadband Internet Access, and other suitable communications services. The communications network 114, in turn, ties the user devices with the early warning system for chronic disease management system server 100.

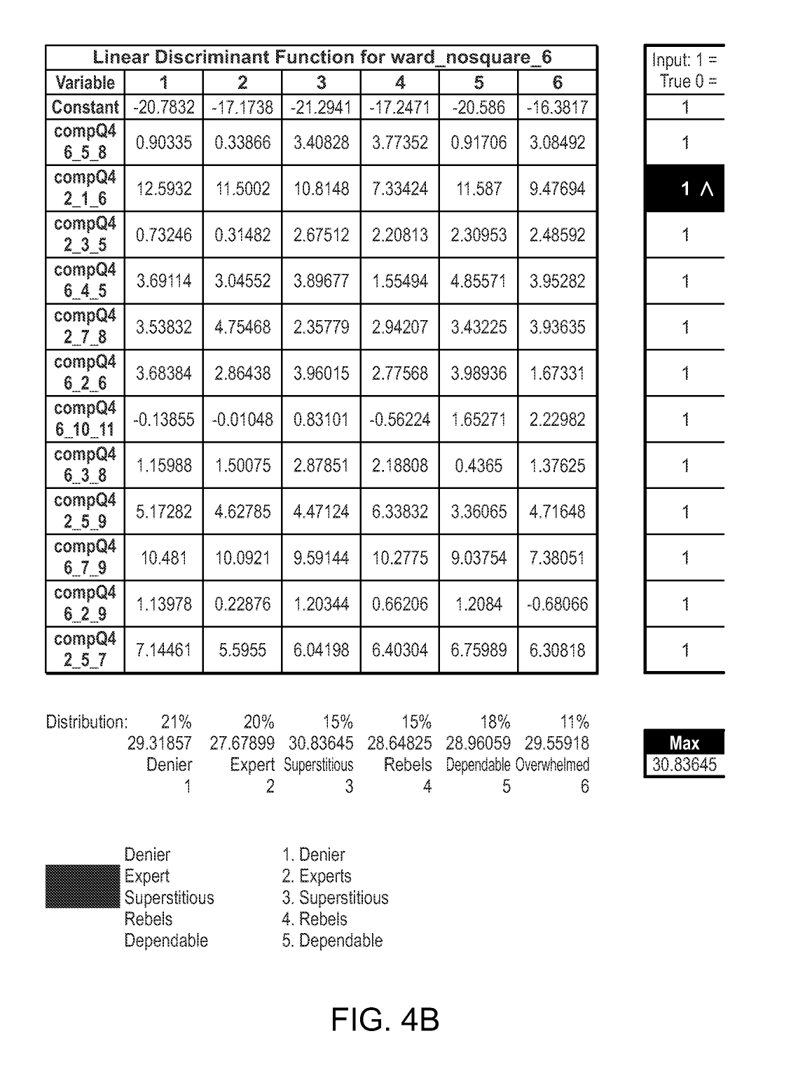

FIG. 6 is a simplified block diagram illustrating exemplary compositional modules of the chronic disease management system 100 in accordance with one or more embodiments. As described previously, this chronic disease management system 100 is in remote communication with a plurality of devices 102, 104, 106, 108, 110, and 112 over a communications network 114.

The event feed from the devices 102, 104, 106, 108, 110, and 112 enters the chronic disease management system 100 via the event parser and queue 202. This module pre-processes each incoming event by authenticating, validating, associating (with the correct patient profile) and subsequently time-stamping that event. It then assigns each event a processing priority in the event queue and sends it forward for processing to the event processing engine 204.

The event-processing engine 204 has two-way communication with the decision support system and look-up tables 206. Events received by the decision support system and look-up tables 206 are acted upon algorithmically, and based on their context the appropriate computations are performed to generate a result which is sent back to the event processing engine 204. The decision support system 206 relies on information from two stores, namely, the profiles store 208 and the event store 210. These two stores will firstly be described below.

The profiles store 208 houses the profiles of individual patients as well as that of groups of patients termed as communities. Note that a community is different from that of a patient care network.

For example, a patient, John, aged 8, has a care network represented by mom, dad, babysitter, grandmother, teacher, coach and school nurse. This represents a single patient and his care network. The end-user in this case is primarily the patient's care network and secondarily the patient himself. In the alternate case where the patient is, say, a 17 year old girl (as opposed to John, the 8 year old boy), the primary end-user would be the patient herself and the secondary ones would be her care network.

On the other hand, a group of patients representing a community could be, for example, a pre-selected group of teens as identified by an interested end-user, say a health insurance company, residing and attending school in a pre-selected set of zip codes, all lying between a pre-selected age range, all “uncontrolled” asthmatics, all with Body Mass Indices greater than 28. This is the profile of a typical community, in which case the end-user is a health insurance company who is trying to tightly monitor this community with the goal of maximizing overall Quality of Life and minimizing healthcare costs.

Hence, both individual patients and communities have profiles stored in the profiles store 208.

In accordance with one or more embodiments, each profile is a vector that comprises a set of scalar features, each of which, in turn, store key data that capture the pattern or signature of the underlying patient or community. Features can be extracted from raw data using a variety of dimensionality reduction algorithms such as principal component analysis, clustering, and curve fitting. Profiles are then transiently manipulated based on temporal feature updates using weighted vector addition methods employing a variety of distance measures such as Euclidean or Mahalanobis.

The profile store 208 also houses person-specific reminders and an actions lookup library. Based on current event input, the current user and the current situation, appropriate reminders and actions are extracted from the profile store and sent to the decision support system and look up tables 206 module for further processing. An example of person-specific reminders and actions is given below.

Reminder: Clean filter of air purifier

Reminder: Put inhaler in backpack

Action: Take 2 puffs of Proventil inhaler 15-30 minutes before exercise

Action: Temp<55F, cover mouth with scarf if outside for more than 10 minutes

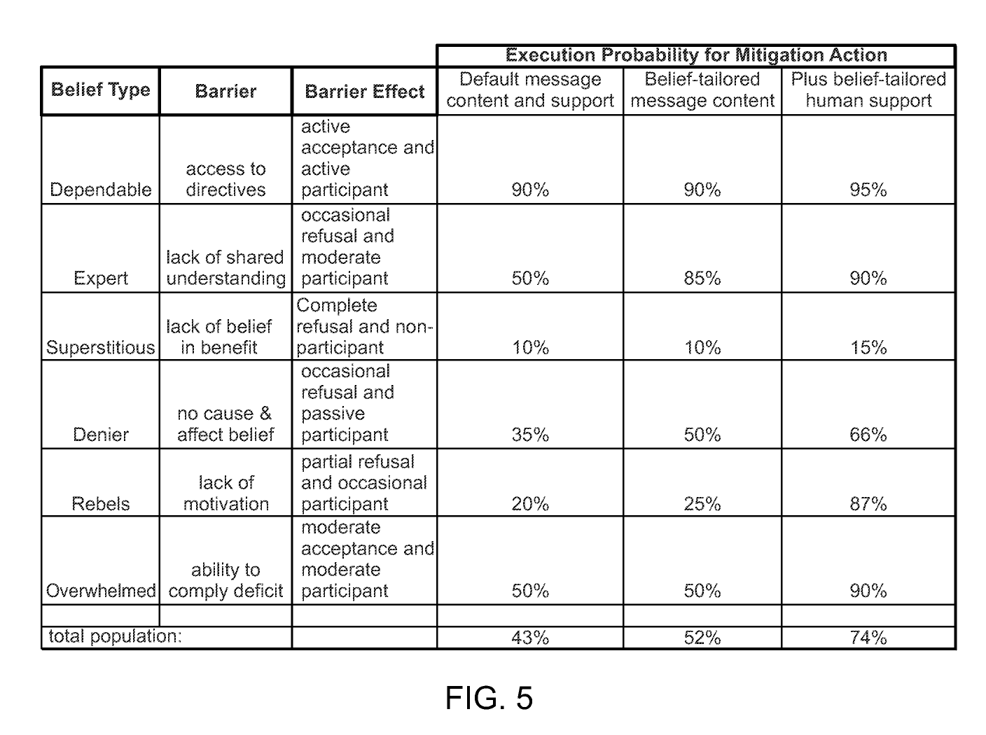

Appropriateness of alert messages are determined by a mitigation message health score from scoring heuristics and ranked by health score and quality of life (QOL) ratings for schedule normalcy and stress of mitigation action message. For example, the four possible messages for mitigation of a risk probability associated with vigorous exercise during moderate mold and cold are listed in the table of FIG. 7. The QOL ranking for messages comprises a normalcy of schedule value (normal=0, disrupts=−2, and eliminates=−4) and a sigma quality of life value (sigma free=0, low stigma=−1, moderate stigma=−2, and high stigma=−4). This ranking is combined with the health score to give a composite message rank value. The system uses this message rank value to determine which message of the possible four messages to send to individuals.

The first two mitigation messages have the same final message value score and are further rank ordered by comparing the aggregate QOL values for each message (0 versus −4) to select the mitigation message with the highest message value with the best QOL value.

In this example, each message comprises message content, message type (personal or generic), message sub-type (action or reminder), message mitigation health score, and message QOL rating (schedule normalcy and stigma).

The system stores all calculated potential messages in the database and these messages are available for reporting purposes. However, we send the messages with the highest rank values to the action dispatcher 212 for distribution via the communication network 114.

The decision support system and look-up tables module 206 is the heart of this system. As mentioned previously, it receives input from the event processing engine 204, processes this input by using appropriate supporting data from the profiles store 208 and the event store 210, to generate an output which is sent back to the event processing engine 204 for further processing and eventual transmission as an action feed back to the user.

An example of the features in a patient profile feature vector could be comprised of, but not limited to, the following=[age, weight, gender, height, home_zipcode, school_zipcode, asthma_severity_assessment_set, asthma_impairment_assessment_set, asthma_control_assessment_set, asthma_triggers_set, asthma_comorbid_conditions_set, recent_disease_history_set, recent_quality_of life_set, patient_local_transient_condition_set].

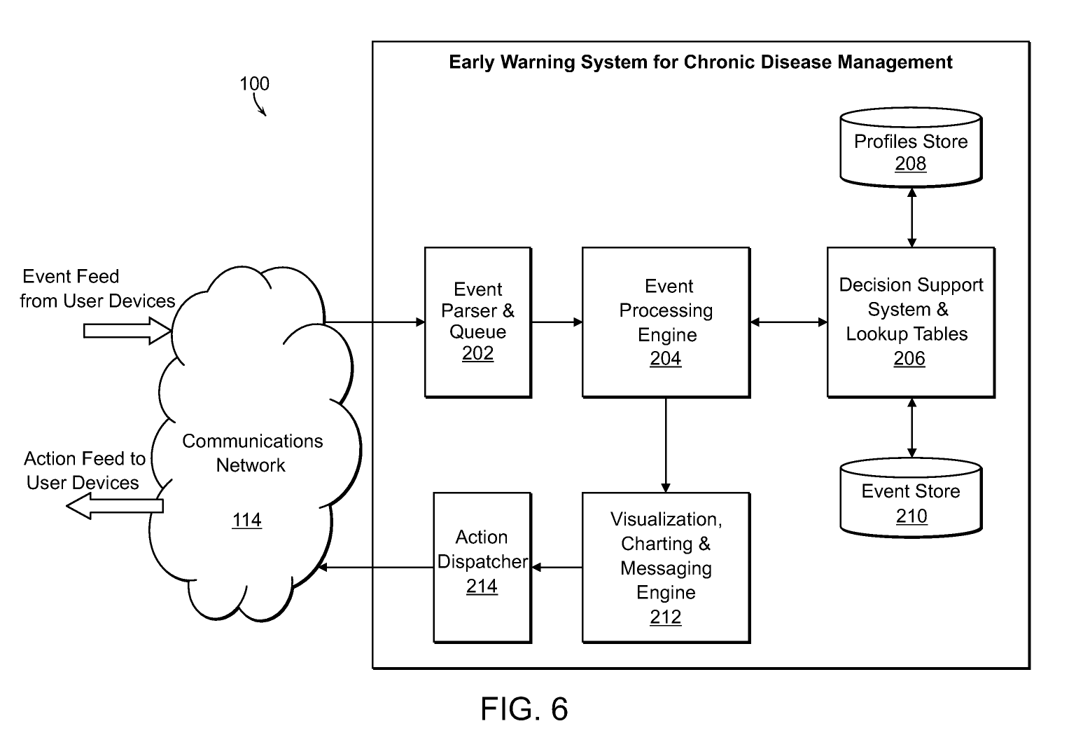

As shown in FIG. 8, the generic profile updating scheme 300 is, New_Patient_Profile 306=function_of(Old_Patient_Profile 302, Transient_Patient_Profile 304), which in FIG. 8 is depicted as simple vector addition.

So consider an example of a partial profile for a patient, i.e., [15 yrs, 110 lbs, male, 64″, 02138, 02239, High, Medium, Medium, Medium, Medium, No, Good, Good] which maps to the profile outline above, i.e., [age, weight, gender, height, home_zipcode, school_zipcode, asthma_severity_assessment_set, asthma_impairment_assessment_set, asthma_control_assessment_set, asthma_triggers_set, asthma_comorbid_conditions_set, recent_disease_history_set, recent_quality_of life_set, patient_local_transient_condition_set].

Now assume that two novel events enter the system for this patient: (1) from the patient's online calendar, which reports that there are scholastic tests at school for the upcoming week; and (2) from the weather service which reports that asthma triggers are spiking for the area where the patient lives.

Based on this new information, two relevant rules fire, i.e., (1) IF the feedback obtained from the patient's school calendar in the current iteration indicates that there are “scholastic tests in the upcoming week”, THEN change this patient's recent_quality_of life_set from Good to Average; and (2) IF the feedback obtained from the weather service regarding the patient's home and school milieu in the current iteration indicates that the “asthma triggers are spiking for that area”, THEN change this patient's asthma_triggers_set from Medium to High.

Note that rules can be crisp, fuzzy, probabilistic or non-probabilistic, with or without temporal components.

Hence the updated profile for that patient is [15 yrs, 110 lbs, male, 64″, 02138, 02239, High, Medium, Medium, High, Medium, No, Average, Good].

Based on this updated profile and the subsequent lowering of the patient's score, a new relevant set of Actions and Reminders are sent to the patient for feedback, for example:

Reminder: (to patient's care network)
Provide a low stress environment to the patient in the upcoming week
Provide positive reinforcement and support
Action: (to patient's care network)
High level of asthma triggers for zipcode xxxx: unobtrusively but closely monitor patient medication regimen adherence

Similarly, an example of the features in a community profile feature vector could be comprised of, but not limited to, the following=[community_zip_set, community_population_summary_statistics_set, community_outbreak_summary_statistics_set, community_disease_trend_set, community_cluster_variation_set].

As shown in FIG. 8, the generic profile updating scheme 300 is, New_Community_Profile 310=function_of(Old_Community_Profile 306, Transient_Community_Profile 308), which in FIG. 8 is depicted as simple vector addition.

The events store 210 houses a longitudinal database of the historical events for the entire universe of patient and community profiles in the system. It is a sub-system that captures the historical event logs of patient or community profile activity. It utilizes industry-standard relational and hybrid object-relationship data models in its design. The goal is to aggregate transactional data into longer time scales to support on-line analytical processing (OLAP) as well as all kinds of statistical analyses, visualization, charting, trending and data-mining. Multiple time-scales of data are also supported to accommodate data ranging from near-real-time trending to data that updates/changes at the daily, weekly, monthly, quarterly, and seasonal frequency. Besides the time/frequency dimension segmentation of data in different databases, there is also the grouping of data items according to the primitive sub-entities that the data items describe at the patient and community levels.

The events store 210 also houses the non-HIPAA content generic reminders and actions library. Based on current event input, the current user and the current situation, the appropriate reminders and actions are extracted from the events store and sent to the decision support system and look up tables 206 module for further processing. An example of reminders and actions is given below.

Reminder: Turn temperature down to 67 degrees in the winter.
Reminder: Use an exhaust fan in kitchens and bathrooms
Reminder: Do not allow smoking in your home, car, or around you.
Reminder: Be sure no one smokes at a child's daycare center or school.
Reminder: Try to stay away from strong odors and sprays, such as perfume, talcum powder, hair spray, paints, new carpet, or particleboard.
Action: High asthma triggers at location zipcode XXXXX
Action: Reduce activity outside due to weather for people with compromised lung function
Action: Take ride to and from school in an air-conditioned vehicle

The decision support system and look-up tables 206 module receives input from the event processing engine 204, processes this input by using appropriate supporting data from the profiles store 208 and the event store 210, to generate an output, which is sends back to the event processing engine 204 for further processing and eventual transmission as an action feed back to the user.

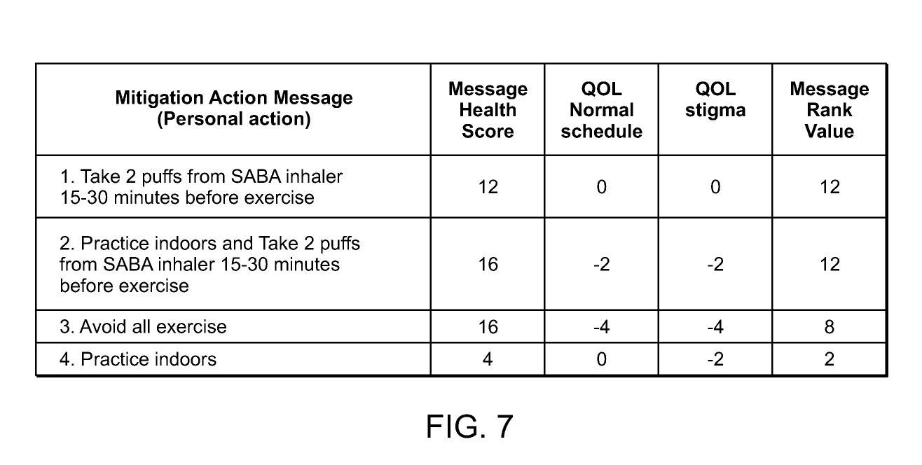

An example comprising selected role-based action messages for a 15 year-old child with asthma and her real world care network (mom and soccer coach) is depicted in a series of iPhone smartphone messages for the child as shown in FIG. 9 and a generic reminder message for the coach depicted as the “#1 Risk Factor” located at the bottom of the iPhone screens depicted in FIG. 9.

In this example, Wendy has mild persistent atopic asthma under control with triggers of exercise, mold allergy, and has three medications (Proventil rescue inhaler, Amanex controller medicine, and Claritin allergy medicine. For this example, Wendy's care network consists of her guardian (Mrs. Wheezer), Wendy, and her soccer coach.

Wendy's personal probability scale is set from her diagnosis at 100 for personal best.

The real world scenario is she is playing an away soccer game tomorrow and the following trigger burdens are identified for her game tomorrow: exercise induced asthma (burden score=12), moderate wind and mold (burden score=6), and air quality poor (burden score=6).

Wendy's starting heuristic health probability score is 98 and after subtracting 24 points, Wendy's probability score (74) places her into the high risk for a putative asthma attack tomorrow.

The potential mitigations based upon her action plan are rank ordered by their mitigation value and the top mitigation actions (along with associated reminders) are identified. Each message has one or more roles assigned to it (guardian, patient, healthcare_advocate, lay_advocate, and sponsor) to identify care network (Wendy, Primary guardian, and coach) distribution targets.

The example messages of FIG. 9 illustrate probable asthma health, action messages, and feedback on actions with concomitant changes in putative asthma health probability graphs.

In accordance with one or more embodiments, the personalized models may be bundled into an applet and installed on the local device (e.g., a smart phone) along with individualized surveys and a subset of personalized data/messages for populating a local data store and operate independently of the network connection to the engine. The local model applet may analyze and respond to subsequent information inputs, feedback, and device inputs without connecting to the full engine, and when network connectivity is available, updating the remote database from the local data store or updating the local personalized model applet by the remote engine.

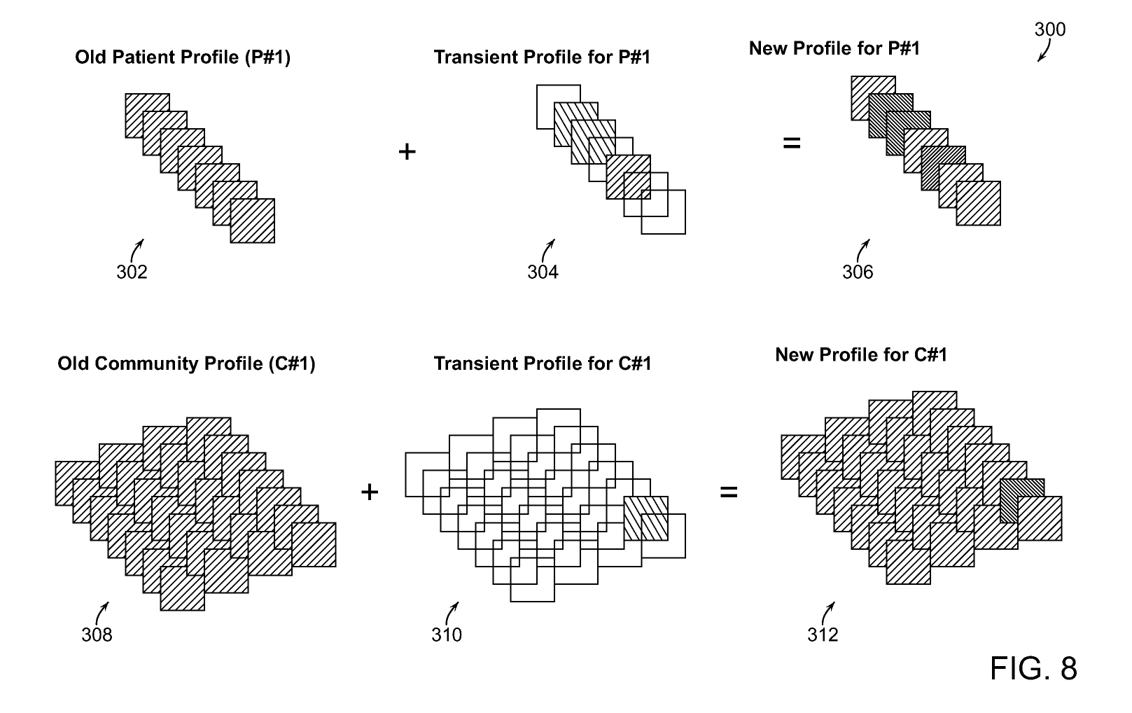

The decision support system and look-up tables 206 module in conjunction with the profiles store 208 and the events store 210, encodes the feedback loop based model predictive control methodology 400 as depicted in FIG. 10.

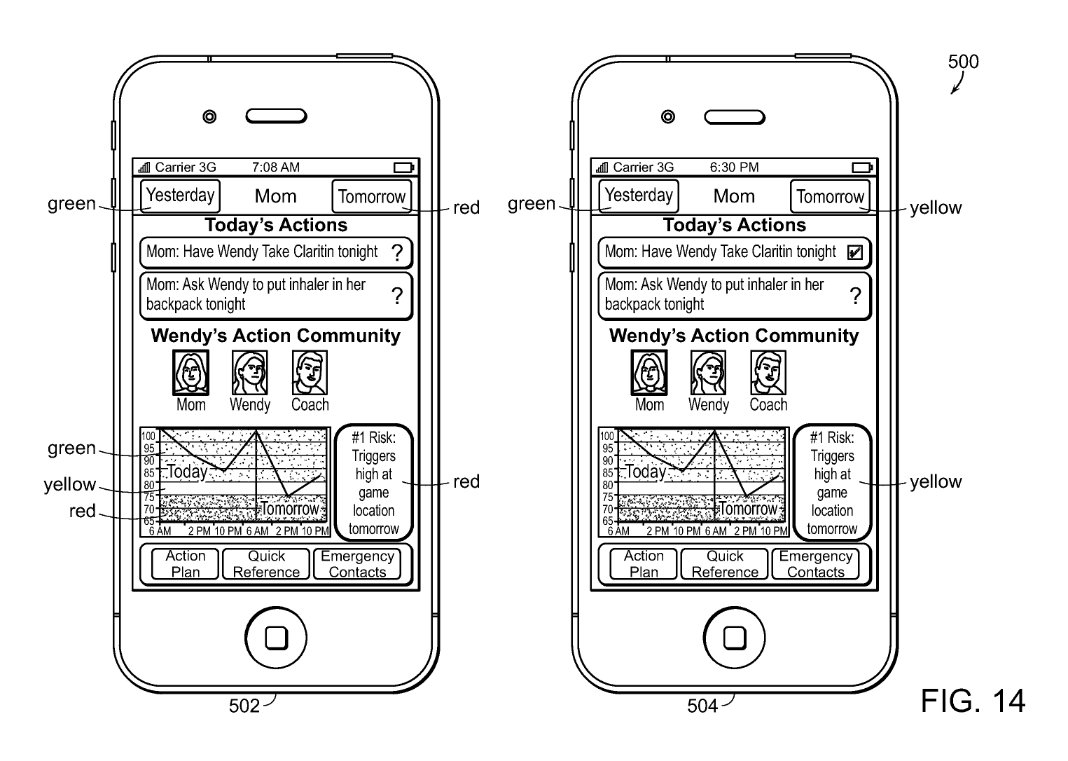

The model predictive control methodology 400 is the fundamental feedback strategy used to provide early warning for chronic disease management. In a specific embodiment, the desired control set-point range, also termed as the total trigger burden for the patient, is first established. For example, in asthma this system utilizes separate asthma impairment (symptoms, SABA use, and pulmonary function) and risk factor (exacerbation frequency, exacerbation severity, and treatment adverse effects) assessments, individual demographics, and trigger sensitivity modifiers to construct a model of an individual's pertinent asthma trigger burdens. Available data inputs from electronic and manually entered sources are used to calculate an individual's trigger burden from each contributing component and individualized trigger burden modifier(s). Trigger burden components have their own statistical calculations and expert rules based upon clinical literature data and knowledge from practitioners treating patients with chronic diseases. These trigger burden calculations are aggregated to build a total trigger burden number that is normalized to a boundary range encompassing healthy normal lifestyle through to a high risk of a disease exacerbation event. This boundary range has three zones: aggregate trigger burdens with no anticipated adverse effect on normal lifestyle, aggregate trigger burdens and trend where behavior should be modified to avoid or reduce further trigger burden addition(s), and aggregate trigger burden that is likely to put the individual at risk of an exacerbation event. In the asthma example, the aggregated trigger burden component numbers are normalized to fit a 25-point scale for ranges of calculations from a good asthma health day to a very poor asthma health day. The total calculated trigger burden is subtracted from an individual's normal “good day” health number (100), which is calibrated against healthy individuals by the asthma severity for that individual, demographics, and the NIH lookup tables. The model is further calibrated so that a test set of scenarios give the appropriate values for predicting a good day (100-80) [green zone], alert range (80-75) [yellow zone], and asthma impairment likely (below 75) [red zone]. An example of personalized range calibration for asthma is given in FIG. 14.

Personalizing the predictive system comprises a series of demographic and diagnostic, and schedule data that are used to establish individual characteristics, their disease profile, and pertinent locations. This information is used by the predictive engine to establish an individualized health probability range, a personalized trigger burden scoring model(s), a personalized trigger mitigation action message set, and a patient's care community actors.

For example, in asthma, the system establishes an individualized reference range by modifying a default health reference range. This range or y-axis comprises a 25 point default scale and is set into three zones: 20 point green zone for low probability of an asthma event, 5 point yellow zone for moderate probability of an asthma event, and below 75 for the red zone representing a high probability for an asthma event.

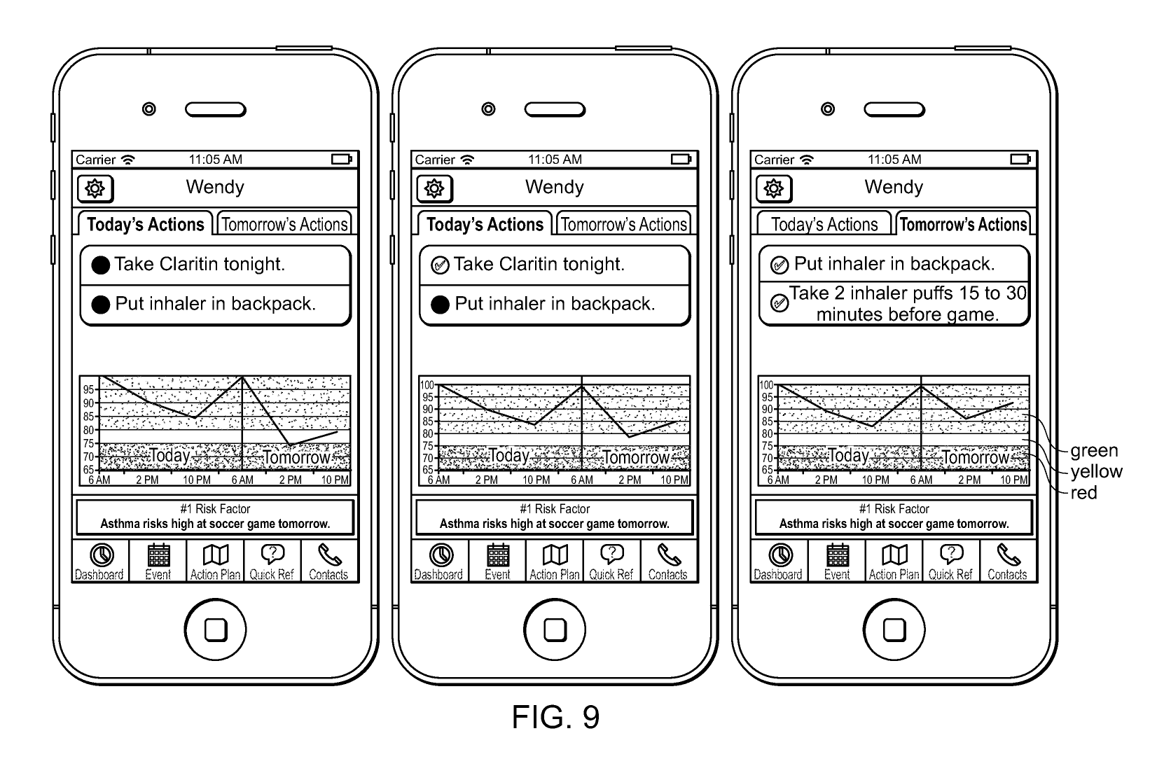

FIG. 11 is a graph illustrating an exemplary asthma probability range and graphed estimate of exacerbation risk.

This default risk range is personalized by asthma control state, co-morbidity, and if known, personal best measurement. For example, in FIG. 11, in the case of asthma, only the top of the green zone is personalized and the other reference range values are fixed. In this graph, green is low probability of an asthma event, yellow is moderate probability of an event, and red is high probability of an event.

Establishing the Y-axis reference range values

Initially establishing range top setting

a. Spirometry determined personal best as a percent of normal lung function.

b. Asthma status of controlled=100

c. Asthma status of uncontrolled=95

### Modifier Examples

a. If patient smokes cigarettes, then default starts at 85
b. If patient has obesity as a co-morbidity, then decrease the 20 point green range by 10% for children, 12% for women, or 8% for men
c. If patient has GERD, then decrease the green+yellow range by 25%
d. If the patient has a respiratory infection, then decrease the green range by 50%

Personalizing the three zones for asthma also use range inputs from

a. Setting the risk ranges based upon doctor recommendations

b. Setting the range using spirometry measurement devices

c. Setting the range zones from patient feedback (yellow=worse symptoms and red=asthma attack). The calculated daily y-axis numbers can be used for plotted score values and their feedback for what risk range is associated with this score value. This is different than using feedback to adjust sensitivity to calculated trigger burdens in the models.

As an example of how to establish range, we will use a patient who has controlled asthma, no recent spirometry measurement of personal best, and has GERD. The top of range is initially set at 100 and we then subtract a quarter of the green range (25-6) to end up with a top of green range of 94.

We use the in one or more embodiments, NAEPP guidelines for the definition of controlled asthma and uncontrolled asthma (FIG. 1b) In these guidelines, children were classified as having uncontrolled asthma if their caregiver reported ANY one of the following criteria: (a) symptoms >2 days per week; (b) awakened by symptoms any night during the past 4 weeks; (c) any activity limitation (in kind or amount) due to impairment or health problem; or (d) rescue inhaler use >5 times per week. All other children were classified as having controlled asthma. (Reference: Assessment of Control in Asthma: The New Focus in Management. S. K. Chhabra; The Indian Journal of Chest Diseases & Allied Sciences, 2008; Vol. 50, 109))

In this example, we use Bayesian methods to calculate the probability of an individual having a respiratory infection in lieu of direct information of an infection. A high probability of respiratory infection during flu and cold season prompts the system to message for asthma action plan identified anti-inflammatory medicines.

The system engine then calculates the aggregate generic asthma trigger burden for relevant locations using either crisp or fuzzy rules, or exposure models using ambient trigger measurements (e.g., air quality, cold, humidity, allergens, and wind). The generic aggregate location trigger burden can be used by the system to message non-PHI (Personal Health Information) warnings to appropriate actors. For example, a coach may receive a message that the aggregate respiratory burden is high at tomorrow's game location and players with asthma should execute asthma action plan directives as appropriate.

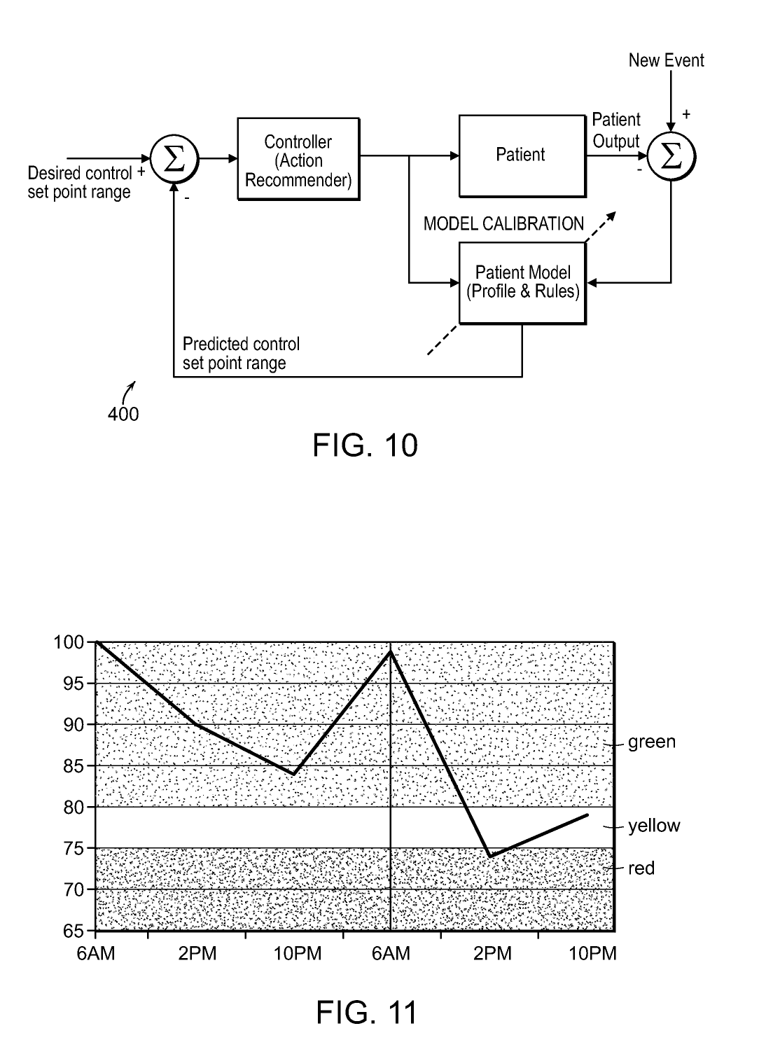

This generic trigger burden is then augmented with person specific information. For example, type of asthma (atopic or non-atopic), duration of scheduled exposure, and vigor of outside activity all modify a generic asthma trigger burden for the specific individual. The exemplary table of FIG. 12 lists the effects of heuristic score and trigger burden reduction based upon these personal factors for ozone induced respiratory burden for people with asthma.

Each trigger burden has a set of action messages recommending an appropriate mitigation behavior. These action messages are rank ordered by their health burden mitigation effect and quality of life impact. The highest value messages are then sent to the appropriate actors as in FIG. 9.

The goal of the controller (action recommender) is to recommend actions to the patient that would minimize the error between the desired control set-point range and the predicted control set-point range and hence keep the patient in the safe range. An individualized set of alert actions and information feedback is used to generate appropriate communication via the control (action recommender) to the asthmatic patient and their care network (family, school, and care provider) to help plan the day so this asthmatic individual aggregate trigger burden stays in the healthy range and does not have a negative trend line predicted to go into the “high risk of exacerbation event” range in the next 24-72 hours of anticipated asthmatic life activities.

This controller (action recommender) resides in the decision support system and lookup tables 206 and employs an ensemble of knowledge engineering and inferencing techniques, fuzzy and expert system rules, Bayesian networks, statistical function approximation methods and flat, multi-dimensional look-up tables.

An example of an expert system rule is given below.

```
IF Home zip is urban, AND,
      IF 0 < Age <= 5 years
            THEN set after school outdoor activities default as
                  Indoor play = 27 h/week
                  Outdoor play = 3 h/week
                  Transit time = 5 h/week
      IF 5 < Age <= 10 years
            THEN set after school outdoor activities default as
                  Indoor play = 12 h/week
                  Outdoor play = 7 h/week
                  Transit time = 7 h/week
      IF 10 < Age <= 17 years
            THEN set after school outdoor activities default as
                  Indoor play = 14 h/week
                  Outdoor play = 5 h/week
                  Transit time = 7 h/week
ELSE report(“Age Out of Range”).
```

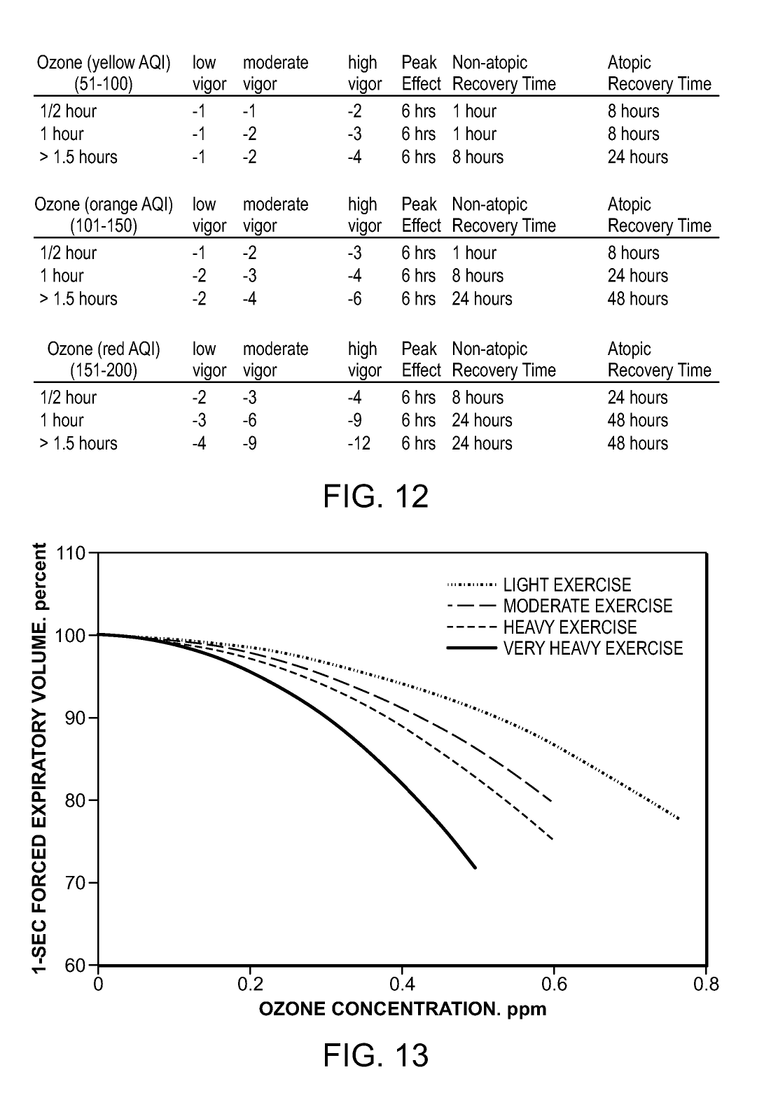

An example of a functional look-up is provided in FIG. 13. Based on the look-up, the relation between the 1-second Forced Expiratory Volume and the Ozone Concentration uses 4 different functions, F1 through F4, based on whether the patient indulges in Very Heavy Exercise, Heavy Exercise, Moderate Exercise, or Light Exercise. In this case, the representation takes the form of an algebraic function. Instead of having a functional representation, one could use a non-functional, flat look-up table (as given in the table of FIG. 12) for the ozone scoring heuristics. This is the second form of representation.

There are also cases where functional or non-functional look up tables are super-imposed by a higher order probabilistic function to incorporate seasonality, i.e., to account for the periodic waxing and waning of the underlying function due to seasonal variations. In this case, all the values in the flat look-up table would be multiplied by a positive or a negative weight, as the case may be, to emphasize or de-emphasize the effect during a certain calendar month as compared to the rest of the year, for a given variable in question such as Ozone (Air Quality Index).

A typical embodiment of the controller (action recommender) includes the following features:
Handling sparse or missing data and the selection of process feature variables that represent the nature of transient operations to capture the underlying process dynamics;
Definition of adjustable parameters in the model and a method of model online tuning;
Monitoring of the system via symptom pertinent manual or automated data feed and device raw data feeds;

Method for periodic assessment of model performance, the application of feedback and the subsequent calibration of the online statistical model.

The actions from the controller are sent both to the patient eventually, via the appropriate delivery mechanisms to a plurality of user devices 102, 104, 106, 108, 110 and to the patient model, i.e., the patient profile, which resides in the profiles store 208.

Based on the actions, the patient generates a new patient output which together with the next new event that is generated results in the prediction of the next predicted control set-point range, which then results in the entire loop repeating itself.

The action feed from the event processing engine 204 is routed to the visualization, charting and messaging engine 212. Given the context of the event inputs, the type of the user and the type of actions generated by the event processing engine 204, the appropriate action feed packet of visuals, trend charts and messages are compiled at this level for onward transmission to the user.

Action dispatcher 214 receives the action packet from the visualization, charting and messaging engine and places it in a queue for transmission to the user. Based on the type of user and the type of user device, it applies the proper configuration wrapper to the action feed packet before its scheduled delivery to the user. FIG. 14 shows an example of an action feed 500 as delivered to a user device. Action feed image 502 shows a screen listing today's actions for patient Wendy, her care group, a chart showing her health number for today and the predicted number for the next day, and the top risk for the next day. Also viewable is a history for the previous day, and a look-ahead screen for the next day. Notice that these buttons are color-coded, in that the previous day is colored green since it was a day where the health number for the patient was in the safe, green zone. Similarly, the button for the next day is colored red, in that, it is predicted that the health number of the patient is expected to dip into the unsafe zone, i.e., the red zone where the risk of asthma exacerbation is high. Also, at any time, Wendy can access her action plan or get to a screen with quick references or her emergency contacts through appropriate buttons. Action feed image 504 shows the effect of patient following the action recommended by action screen 502 (i.e., take a medication, namely, Claritin). Notice that the completion of this action and the appropriate feedback communication of the same through the check-box, causes a change in the prediction for the upcoming day, in that the health number is now predicted to rise to the moderate risk yellow zone from the previously predicted, high risk red zone.

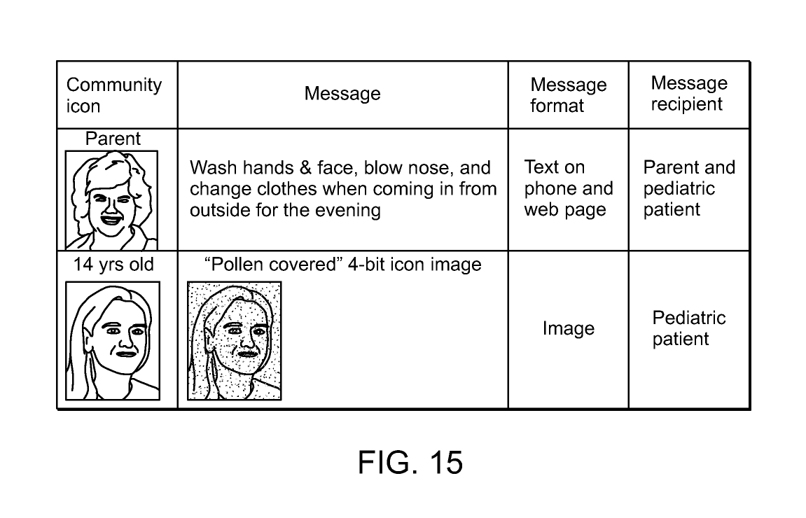

Messages sent to the patient may be comprised of text, voice, video, and/or images. The recipient may select their favored message form factor (for example, voice versus text). Additionally, the system may select the form factor and content. For example, message content and format change is selected by the system for the message associated with high outside allergen exposure to allergen sensitive patients. A directive to the mom to stop dosing the allergen once inside is: “Wash hands & face, blow nose, and change clothes when coming in from outside for the evening.” This directive message is sent to the parent as text when the patient is 6 years old. However, this message is also sent to the patient if they are 14 years old. The message sent to the 14 year old is accompanied by an image comprising a bit reduced image of their community icon picture signifying their undesirable pollen-covered body to incent them to execute the recommended action (example in FIG. 15).

Message content may also be tailored to the belief type of the recipient. For example, using the Ward's Belief survey to type message recipient belief, a person with the dependable belief profile receives the directive message whereas a person with the expert belief type receives the directive message and a reference link to additional content or URL to a trusted expert source that goes into why this action is recommended (e.g., http://www.webmd.com/asthma/guide/asthma-treatment-care for asthma).

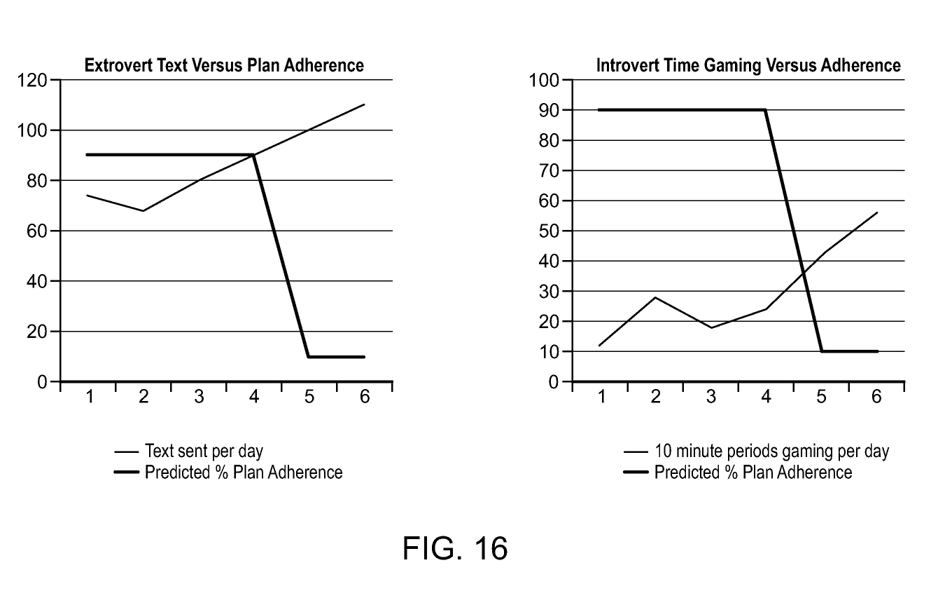

Another aspect to messaging is to understand, predict and account for the type of feedback expected from a patient in response to a message sent to them by the system. The engine may receive information about behaviors that are modeled on a population probability and on an individual longitudinal basis to establish risk and associated mitigation messages. For example, phone usage and evening online game activity comprise a model that predicts risk of a holiday from treatment plan adherence in teenagers and young adults. The example below comprises monitoring the number of text messages from an extroverted cystic fibrosis patient and monitoring the number of hours on an online game for an introverted cystic fibrosis patient. The two exemplary charts in FIG. 16 show the case of an introverted versus an extroverted teen, both of whom need to be handled separately to increase the probability of maximizing the chances of regaining their adherence to their regimen.

In the case of the extrovert, a >20% increase in texting sustained for more than one day is associated with a marked decrease in the probability of disease management plan adherence. Notifying the extrovert's care community of a high risk of non-adherence can result in successful intervention to bring this individual into adherence.

In the case of the introvert, doubling the number of 10 minute gaming periods per weekday is correlated with an increased risk for non-adherence. This information can be sent to their care community prompting an appropriate intervention to bring them into adherence.

The utilization of such probabilistic sub models is one aspect of this system.

Another aspect of the user interface system is education, which is both a component of alerts and a response to feedback. In accordance with one or more embodiments, an education component is accessible by the user via both the website as well as the personal mobile device. It is important to train the family, the child, and others not associated with the medical community on how to use medicines, what to do about trigger burden, and how to manage a home-based medically safe place(s). For example, periodic alerts on how to manage the home-based medically safe place(s) allows the model to correctly score the positive effect of allowing the asthmatic child's body to reduce the body's dose of triggers and to minimize reintroduction of triggers into the home-based medically safe place. For example, people exposed to allergens should wash their hands, blow their nose, and if warranted, change clothes to stop reintroducing allergens into their system and their home-based medically safe place. Additionally, changing filters on HEPA air cleaners and bags on HEPA vacuum cleaners can be important to maintain the proper modifier on the positive effect of the medical safe place.

Another example of the importance of education would be the proper use of an inhaler. The model assumes an inhaler dose of 50% of medicine delivered to the lungs. Poor use of inhaler can eliminate more than 90% of the dose from reaching deep into the lungs. This error can distort the predictive model because the positive effect of the inhaler is over calculated in the model. Additionally, failure to inhale the whole dose of the medicine may be interpreted as poor asthma control and the doctor may unnecessarily increase the dose of medication leading to more drug side effects and/or long-term health effects in children.

For example, in asthma education components can include:

1. Use of the inhaler and long acting control medications
2. Greater emphasis on the two aspects of the written asthma action plan—(1) daily management, and (2) how to recognize and handle worsening asthma.
3. Home medical safe space instruction.
4. Importance of proper action to reducing trigger burden.
5. If using a device, how to use the device and how to read the device.
6. Interpreting the written action plan.

Hence, there are three basic process flows to consider, namely, 1. when triggered by a patient accessing the system, 2. when triggered by the occurrence of a new event, and, 3. when triggered by the occurrence of a pre-determined time-of-day. Once the personalized model is set up with patient demographics, disease diagnosis, treatment plan, and recurring individual schedule (comprising home, work, and school schedule), the system is able to predict and send warning messages with mitigation recommendations without further input or feedback from a patient or their care network for extended periods of time (for days to months). In the asthma example, these personalized default models are able to predict and make recommendations on trigger burdens accounting for one-third of exacerbation events in children with asthma.

In the first case the process flow is triggered when a patient accesses the system (pull) to either report a new event or to simply get the most current feedback from the system. If this patient happens to be a new patient, then the patient history questionnaire and the patient axis calibration modules are executed at the outset for the creation of this patient's customized profile and healthy set-point score range. If the patient is not new but has accessed the system to self-report a new event, then that patient's profile is accessed, a new score is computed and a new report (with customized reminders, actions, visualizations, education snippets, trends and statistics) based on the patient's current score, is generated and transmitted to that patient.

In the second case, the process flow is triggered simply by the occurrence of a new event. Upon occurrence of this new event and the reporting of the same to the system, a process retrieves all the patient and community profiles in the database that are affected by this event. These profiles are then updated based on the new event and scores are generated for each of the retrieved patient profiles and the corresponding reports are “pushed” to each of the patients. In case of the relevant community profiles, the respective profiles are updated but no scores are generated. Instead, the community report is generated and reported back to the appropriate owner of that profile (example an insurance company).

In the third case, the process flow is triggered by the occurrence of a pre-determined time of day. Based on the needs it is determined, a priori to run a computation and generate reports for specific sets of patients or communities. For example, a patient might request reports back at noon every Sunday (so they can plan their school week) or a health insurance company might request a report on a specific group of their patients (community) that subscribe to a specific type of health plan at the end of every quarter. At the occurrence of these pre-determined times and dates, a process retrieves all the relevant patient and community profiles. No profiles are updated and no scores are generated, but the relevant reports are generated and transmitted to the respective owners.

In accordance with one or more further embodiments, an incentive marketplace can be provided for patients and caregivers to promote certain behaviors by patients and caregivers.

A reverse auction system can be provided to allow care providers and care guardians (e.g. parents, schools, etc.) to create:

1. Incentives to have patients modify behavior to be in line with best health practices and treatment action plans (e.g., moderate exposure to exacerbation triggers).
2. Incentives for care guardians to advocate for their patient to execute against treatment plan. For example, a parent might get a coupon for Walmart or other reward if he or she keeps up the child's event calendar or adds surrogate guardians into the alert pool (e.g., a parent hosting a sleepover). In another example, the asthmatic child's parent can create an incentive for an older teen to buddy with their asthmatic child, guiding them into self reliance in executing their asthma action plan.

In one or more embodiments, social network analysis is used to determine access and survey effectiveness of professional care providers and guardians/buddies. The use of social network analysis (SNA) to identify communication hubs is well understood. Using SNA to profile which care givers are frequently contacted (phone, meeting, email, text, etc.) can be used to profile nurse advocates and other professional care givers' accessibility to patients and their guardians. Additionally, this analysis can be used to profile accessibility of volunteers who “buddy” with patients and their guardians to help them learn and execute care plans and healthy behaviors.

The accessibility measure can be used to select and measure these people's effectiveness by other means (e.g., survey, data on increased healthy behavior, etc.).

In one or more embodiments, the SNA is used to profile access and effectiveness of care givers and care advocates and correlated with reduction of bad health events. This is especially important in chronic diseases such as asthma where access to education and advice can make a big difference in outcomes. As teen asthmatics begin to pull away and become independent from their parents and guardians, this capability to monitor and measure access to “peer asthma experts” can be very important. A volunteer, e.g., an older experienced asthmatic teen, would have a lot of credibility to a younger teen beginning to become independent from parents and guardians. The SNA tools can be used to determine which volunteers and educators are truly accessible to patients and others in the patient network. Additionally, the SNA data can be used to better measure effectiveness of these people. For example, the number of consults with the trainer compared to the trend number of bad asthma events is a measure of effectiveness of the care network worker or volunteer's effectiveness at education.

The satisfaction of the communication event by both parties is a measure of the likelihood of repeat connections in the future for advice or education. A system in accordance with one or more embodiments can channel future communication to the care advocates that score higher for effective connections.

The techniques described above are preferably implemented in software, and accordingly one of the preferred implementations of the invention is as a set of instructions (program code) in a code module resident in the random access memory of a programmable computer. Until required by the computer, the set of instructions may be stored in another computer memory, e.g., in a hard disk drive, or in a removable memory such as an optical disk (for eventual use in a CD or DVD ROM) or floppy disk (for eventual use in a floppy disk drive), a removable storage device (e.g., external hard drive, memory card, or flash drive), or downloaded via the Internet or some other computer network. In addition, although the various methods described are conveniently implemented in a general purpose computer selectively activated or reconfigured by software, one of ordinary skill in the art would also recognize that such methods may be carried out in hardware, in firmware, or in more specialized apparatus constructed to perform the specified method steps.

Having thus described several illustrative embodiments, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one embodiment are not intended to be excluded from similar or other roles in other embodiments. Additionally, elements and components described herein may be further divided into additional components or joined together to form fewer components for performing the same functions. Accordingly, the foregoing description and attached drawings are by way of example only, and are not intended to be limiting.

What is claimed is:

1. A computer-implemented method for assisting a plurality of patients manage chronic health conditions, for each patient the method comprising:
(a) receiving information from the patient or a member of a patient care network on an expected patient activity at a given future time period;
(b) determining expected transient local ambient conditions in the patient's surroundings during the expected patient activity at the given future time period;
(c) predicting health exacerbations for the patient using a stored computer model of the patient based on a desired patient control set-point range, the expected patient activity, and the expected transient local ambient conditions; and
(d) proactively sending a message to the patient or a member of the patient care network before the given future time period, the message alerting the patient or a member of the patient care network of the predicted health exacerbations for the patient and identifying one or more corrective actions for the patient to avoid or mitigate the predicted health exacerbations.

2. The method of claim 1, further comprising calibrating the computer model of the patient based on the validity of the predicted health exacerbations, patient behavior modification success, longitudinal health trends for the patient, or aliased learnings from patients with similar profiles, using first principles from research literature or using heuristic knowledge from domain experts.

3. The method of claim 1, further comprising determining longitudinal health trends for the patient and transmitting reports on the health trends to the patient or a member of the patient care network.

4. The method of claim 1, further comprising determining aggregated longitudinal health trends for a community of patients and transmitting reports on the health trends of the community to another party.

5. The method of claim 4, wherein said another party comprises a healthcare administrator, a healthcare network, a healthcare payer, a guardian, a surrogate guardian, a lay advocate, a disease management advocate, an insurance company, or a governmental agency.

6. The method of claim 1, wherein the computer model of the patient includes a patient profile including data on the medical condition of the patient obtained as clinical data from physical exams, from laboratory tests, or as collected using input devices, condition-relevant exacerbation triggers associated with the patient, a physician-provided management plan for the patient, or sociological and demographic data associated with the patient.

7. The method of claim 1, further comprising periodically collecting data from one or more input devices operated by the patient or a member of the patient care network to, develop a customized baseline feature vector for the patient using physiological criteria, monitor for deviations in the baseline, and generate a score based on the deviations.

8. The method of claim 7, wherein the score is generated based on amplitudes and frequencies of various features in a feature vector.

9. The method of claim 1, further comprising developing an alert action plan for each of the plurality of patients or a member of a patient's care network, periodically updating the plan based on burden measures, and reporting the alert action plan to the patient or a member of the patient care network, wherein the action plan is customized to generally minimize error between the desired patient control set-point range and a predicted control set-point range, to keep the patient in a wellness and health management safe range.

10. The method of claim 1, further comprising determining a belief and personality type of the patient based on a priori population segmentation methods in research literature, and tailoring the message based on the patient's belief and personality type.

11. The method of claim 1, wherein the transient local conditions comprise local air quality, allergen levels, temperature, chemicals, humidity, wind, prevailing atmospheric conditions, indoor environmental conditions, or transient localized comorbidity disease outbreak conditions.

12. The method of claim 1, wherein the chronic disease comprises a disease selected from the group consisting of Acquired Immune Deficiency Syndrome (AIDS), Attention Deficit/Hyperactivity Disorder (ADHD), Allergies, Amyotrophic Lateral Sclerosis (ALS), Alzheimer's Disease, Arthritis, Asthma, Behcet's syndrome, Bipolar Disorder, Bronchitis, Cardiomegaly, Cardiomyopathy, Crohn's disease, Chronic cough, Chronic Fatigue Syndrome (CFS), Chronic Obstructive Pulmonary Disease (COPD), Congestive Heart Failure, Cystic Fibrosis, Depression, Diabetes, drug addiction, alcohol addiction, Emphysema, Fibromyalgia, Gastroesophageal reflux disease (GERD), Gout, Hansen's Disease, Hunter syndrome, Huntington's disease, Hypertension, Marfan syndrome, Mesenteric lymphadenitis, Multiple Sclerosis, Migraines, Myelofibrosis, Nephrotic syndrome, Obesity, Parkinson's disease, Pneumoconiosis (interstitial lung diseases), Pulmonary edema, Pulmonary Fibrosis, Pulmonary hypertension, Reactive airway disease, Sarcoidosis, Scleroderma, Systemic Lupus Erythematosus, and Ulcerative colitis

13. The method of claim 1, further comprising utilizing incentive schemes to encourage patients to modify their behavior to be in line with best health practices and treatment action plans, or to assist care guardians advocate for their patient to execute against a patient treatment plan.

14. The method of claim 1, further comprising educating the patient and members of the patient care network about patient disease management utilizing patient alerts or responses to patient feedback.

15. The method of claim 1, further comprising utilizing social network techniques to mine longitudinal patient data across multiple segmented categories of patients to better understand and optimize disease management tenets and their effectiveness.

16. An early warning system for assisting a plurality of patients manage chronic health conditions, the early system comprising a computer system communicating with client devices operated by the plurality of patients over a communications network, for each patient the computer system being configured to:
(a) receive information from the patient or a member of a patient care network on an expected patient activity at a given future time period;
(b) determine expected transient local ambient conditions in the patient's surroundings during the expected patient activity at the given future time period;
(c) predict health exacerbations for the patient using a stored computer model of the patient based on a desired patient control set-point range, the expected patient activity, and the expected transient local ambient conditions; and
(d) proactively transmit a message to the patient or a member of the patient care network before the given future time period, the message alerting the patient or a member of the patient care network of the predicted health exacerbations for the patient and identifying one or more corrective actions for the patient to avoid or mitigate the predicted health exacerbations.

17. The early warning system of claim 16, wherein the computer system is further configured to calibrate the computer model of the patient based on the validity of the predicted health exacerbations, patient behavior modification success, longitudinal health trends for the patient, or aliased learnings from patients with similar profiles, using first principles from research literature or using heuristic knowledge from domain experts.

18. The early warning system of claim 16, wherein the computer system is further configured to determine longitudinal health trends for the patient and transmit reports on the health trends to the patient or a member of the patient care network.

19. The early warning system of claim 16, wherein the computer system is further configured to determine aggregated longitudinal health trends for a community of patients and transmit reports on the health trends of the community to another party.

20. The early warning system of claim 19, wherein said another party comprises a healthcare administrator, a healthcare network, a healthcare payer, a guardian, a surrogate guardian, a lay advocate, a disease management advocate, an insurance company, or a governmental agency.

21. The early warning system of claim 16, wherein the computer model of the patient includes a patient profile including data on the medical condition of the patient obtained as clinical data from physical exams, from laboratory tests, or as collected using input devices, condition-relevant exacerbation triggers associated with the patient, a physician-provided management plan for the patient, or sociological and demographic data associated with the patient.

22. The early warning system of claim 16, wherein the computer system is further configured to periodically collect data from one or more input devices operated by the patient or a member of the patient care network to, develop a customized baseline feature vector for the patient using physiological criteria, monitor for deviations in the baseline, and generate a score based on the deviations.

23. The early warning system of claim 7, wherein the score is generated based on amplitudes and frequencies of various features in a feature vector.

24. The early warning system of claim 16, wherein the computer system is further configured to develop an alert action plan for each of the plurality of patients or a member of a patient's care network, periodically update the plan based on burden measures, and report the alert action plan to the patient or a member of the patient care network, wherein the action plan is customized to generally minimize error between the desired patient control set-point range and a predicted control set-point range, to keep the patient in a wellness and health management safe range.

25. The early warning system of claim 16, wherein the computer system is further configured to determine a belief and personality type of the patient based on a priori population segmentation methods in research literature, and tailoring the message based on the patient's belief and personality type.

26. The early warning system of claim 16, wherein the transient local conditions comprise local air quality, allergen levels, temperature, chemicals, humidity, wind, prevailing atmospheric conditions, indoor environmental conditions, or transient localized comorbidity disease outbreak condition.

27. The early warning system of claim 16, wherein the chronic disease comprises a disease selected from the group consisting of Acquired Immune Deficiency Syndrome (AIDS), Attention Deficit/Hyperactivity Disorder (ADHD), Allergies, Amyotrophic Lateral Sclerosis (ALS), Alzheimer's Disease, Arthritis, Asthma, Behcet's syndrome, Bipolar Disorder, Bronchitis, Cardiomegaly, Cardiomyopathy, Crohn's disease, Chronic cough, Chronic Fatigue Syndrome (CFS), Chronic Obstructive Pulmonary Disease (COPD), Congestive Heart Failure, Cystic Fibrosis, Depression, Diabetes, drug addiction, alcohol addiction, Emphysema, Fibromyalgia, Gastroesophageal reflux disease (GERD), Gout, Hansen's Disease, Hunter syndrome, Huntington's disease, Hypertension, Marfan syndrome, Mesenteric lymphadenitis, Multiple Sclerosis, Migraines, Myelofibrosis, Nephrotic syndrome, Obesity, Parkinson's disease, Pneumoconiosis (interstitial lung diseases), Pulmonary edema, Pulmonary Fibrosis, Pulmonary hypertension, Reactive airway disease, Sarcoidosis, Scleroderma, Systemic Lupus Erythematosus, and Ulcerative colitis

28. The early warning system of claim 16, wherein the computer system is further configured to provide incentives to encourage patients to modify their behavior to be in line with best health practices and treatment action plans, or to assist care guardians advocate for their patient to execute against a patient treatment plan.

29. The early warning system of claim 16, wherein the computer system is further configured to provide education the patient and members of the patient care network about patient disease management utilizing patient alerts or responses to patient feedback.

30. The early warning system of claim 16, wherein the computer system is further configured to utilize social network techniques to mine longitudinal patient data across multiple segmented categories of patients to better understand and optimize disease management tenets and their effectiveness.
